(19) United States
(12) Patent Application Publication
(10) Pub. No.: US 2023/0190227 A1
(43) Pub. Date: Jun. 22, 2023

(54) PLAQUE BURDEN INDICATION ON LONGITUDINAL INTRALUMINAL IMAGE AND X-RAY IMAGE

(71) Applicant: PHILIPS IMAGE GUIDED THERAPY CORPORATION, San Diego, CA (US)

(72) Inventors: Asher COHEN, SAN DIEGO, CA (US); Pei-Yin CHAO, EINDHOVEN (NL); Ronald Christiaan HELMSTRIJD, EINDHOVEN (NL)

(21) Appl. No.: 18/075,561

(22) Filed: Dec. 6, 2022

Related U.S. Application Data
(60) Provisional application No. 63/290,483, filed on Dec. 16, 2021.

(51) Int. Cl. A61B 8/12; A61B 8/08

(52) U.S. Cl. CPC A61B 8/12; A61B 8/0891; A61B 8/5207

(57) ABSTRACT

A system includes a processor circuit that receives a plurality of intraluminal images obtained by an intraluminal imaging device. The processor circuit determines a plaque burden for each of the plurality of intraluminal images and identifies a region of the body lumen within an image of the body lumen. The processor circuit then outputs to a display the image of the body lumen, a first plaque burden value corresponding to a distal end of the region, and a second plaque burden value corresponding to a proximal end of the region.

### CROSS-REFERENCE TO RELATED APPLICATIONS

This application claims priority to and the benefit of U.S. Provisional Application No. 63/290,483, filed Dec. 16, 2021, which is incorporated by reference herein in its entirety.

### TECHNICAL FIELD

The present disclosure relates generally to co-registering data from different medical diagnostic modalities. In particular, a longitudinal plaque burden indication is automatically generated and displayed along a longitudinal intraluminal image and an x-ray image.

### BACKGROUND

Physicians use many different medical diagnostic systems and tools to monitor a patient's health and diagnose medical conditions. Different modalities of medical diagnostic systems may provide a physician with different images, models, and/or data relating to internal structures within a patient. These modalities include invasive devices and systems, such as intravascular systems, and non-invasive devices and systems, such as x-ray systems, and computed tomography (CT) systems. Using multiple diagnostic systems to examine a patient's anatomy provides a physician with added insight into the condition of the patient.

In the field of intravascular imaging, co-registration of data from invasive devices (e.g., intravascular ultrasound (IVUS) devices or instantaneous wave-free ratio (iFR) devices) with images collected non-invasively (e.g., via x-ray angiography) is a powerful technique for improving the efficiency and accuracy of vascular catheterization procedures. Co-registration identifies the locations of intravascular data measurements along a blood vessel by mapping the data to an angiography image of the vessel. A physician may then know exactly where in the vessel a measurement was made, rather than estimate the location.

A plaque burden is a metric of how much of a cross-sectional area of a vessel is composed of an obstruction, like plaque. Plaque burden measurements assist a physician in understanding the flow characteristics of a vessel and the extent of a blockage. Plaque burden measurements may be determined based on an IVUS image. Through co-registration, plaque burden measurements may be mapped to an angiography image of the vessel or a longitudinal view of the vessel. A display of plaque burden measurements along a region of the vessel to be treated may assist a physician in determining proper stent placement. However, for some imaging procedures, the operator manually quantifies plaque burden values at a stent's proximal and distal markers. Manually quantifying plaque burden values adds significant time to the procedure, impacting the adoption of the application of plaque burden measurements to stent-edge positions. In addition, plaque burden measurements are typically displayed numerically. Numerical plaque burden measurements may require unnecessary and timely analysis by a physician. Requiring a physician to compare multiple numerical plaque burden measurements for a region of the vessel may increase the length of an imaging or treatment procedure and lead to errors in the placement of a treatment device.

### SUMMARY

Embodiments of the present disclosure are systems, devices, and methods for generating a longitudinal plaque burden indication on a longitudinal intraluminal image and an extraluminal image. Aspects of the present disclosure may advantageously provide a physician with a graphic view of plaque burden measurements at multiple locations along a vessel simultaneously. As a result, the physician may more quickly and accurately determine whether a proposed stent location is properly placed or need adjustment. In one aspect, multiple x-ray images are acquired showing a vessel of a patient. As these x-ray images are acquired, an IVUS imaging device is moved through the vessel acquiring IVUS images. Each IVUS image is then associated with the location at which it was received. Each IVUS image is analyzed to identify a vessel wall and lumen boundary. Based on these identified features, a plaque burden value is calculated as a percentage of plaque area verses vessel wall area.

A longitudinal plaque burden graphic is also generated. The longitudinal plaque burden graphic may include multiple segments. Each segment corresponds to one or more IVUS images. Each segment may include one or more regions. One region corresponds to a luminal area of an IVUS image and an additional region corresponds to an area of plaque or other obstruction. The size of the region corresponding to luminal area graphically illustrates the luminal area value and the size of the region correspond to plaque area graphically illustrates the plaque area value. In this way, a physician may see the extent of plaque verses lumen area quickly and easily by comparing the relative sizes of the regions of a segment. When multiple segments are displayed, a physician may compare the extent of plaque along a vessel quickly and easily.

The longitudinal plaque burden graphic may be displayed to a user in conjunction with a longitudinal intravascular image or an extraluminal image, like an x-ray image. The longitudinal plaque burden graphic may be displayed during an imaging procedure, during a planning procedure, or during a treatment procedure. The longitudinal plaque burden graphic can be displayed for a region of a vessel where a stent is intended to be placed. In this case, as a user adjusts the location of either end of the planned stent, the longitudinal plaque burden graphic may be updated in real time to provide a graphic representation of the extent of plaque along the intended location of the stent.

In an exemplary aspect, a system is provided. The system includes a processor circuit configured for communication with an intraluminal imaging device, wherein the processor circuit is configured to: receive a plurality of intraluminal images obtained by the intraluminal imaging device during the movement of the intraluminal imaging device within a body lumen of a patient; determine a plaque burden for each of the plurality of intraluminal images; identify a region of the body lumen in an image of the body lumen; output, to a display in communication with the processor circuit, a screen display comprising: the image of the body lumen; a first plaque burden value corresponding to a distal end of the region; and a second plaque burden value corresponding to a proximal end of the region, wherein the first plaque burden value and the second plaque burden value are positioned proximate to the image of the body lumen.

In one aspect, the image of the body lumen comprises a longitudinal view of the body lumen. In one aspect, the processor circuit is further configured to generate a plaque burden indicator corresponding to the region. In one aspect, the plaque burden indicator includes one or more segments, each segment corresponding to one or more intraluminal images of the plurality of intraluminal images. In one aspect, each segment includes one or more regions, the visual appearance of the one or more regions corresponding to a cross-sectional area of the body lumen or a cross-sectional area of plaque. In one aspect, the plaque burden indicator is positioned within the screen display such that the plaque burden indicator is aligned with the region in the longitudinal view. In one aspect, the processor circuit is further configured to determine a recommended position for a treatment device based on the plaque burden of one or more of the plurality of intraluminal images. In one aspect, the first plaque burden value corresponds to a distal end of the treatment device and the second plaque burden value correspond corresponds to a proximal end of the treatment device. In one aspect, the processor circuit is further configured to: receive, by an input device, a user input moving at least one of the proximal end of the region or the distal end of the region to a different location; and in response to the user input, update at least one of the first plaque burden value or the second plaque burden value based on the different location. In one aspect, the processor circuit is further configured for communication with an extraluminal imaging device and the image of the body lumen comprises the extraluminal image. In one aspect, the processor circuit is further configured to coregister the plurality of intraluminal images and the plaque burden for each of the plurality of intraluminal images to corresponding positions within an extraluminal image. In one aspect, the processor circuit is further configured to generate a plaque burden indicator corresponding to the region. In one aspect, the plaque burden indicator includes one or more segments, each segment corresponding to one or more intraluminal images of the plurality of intraluminal images. In one aspect, each segment includes one or more regions, the visual appearance of the one or more regions corresponding to a cross-sectional area of the body lumen or a cross-sectional area of plaque. In one aspect, the plaque burden indicator is positioned within the screen display such that the plaque burden indicator aligns with the region in the extraluminal image.

In an exemplary aspect, a method is provided. The method includes receiving, with a processor circuit in communication with an intraluminal imaging device, a plurality of intraluminal images obtained by the intraluminal imaging device during a movement of the intraluminal imaging device within a body lumen of a patient; determining, with a processor circuit, a plaque burden for each of the plurality of intraluminal images; identifying, with a processor circuit, a region of the body lumen in an image of the body lumen; outputting, to a display in communication with the processor circuit, a screen display comprising: the image of the body lumen; a first plaque burden value corresponding to a distal end of the region; and a second plaque burden value corresponding to a proximal end of the region, wherein the first plaque burden value and the second plaque burden value are positioned proximate to the image of the body lumen.

In an exemplary aspect, a system is provided. The system includes an intravascular imaging device; and a processor circuit configured for communication with the intravascular imaging device, wherein the processor circuit is configured to: receive a plurality of intravascular images obtained by the intravascular imaging device during the movement of the intravascular imaging device within a blood vessel of a patient; determine a plaque burden for each of the plurality of intravascular images; identify a region of the blood vessel in an image of the blood vessel; generate a plaque burden indicator corresponding to the region of the blood vessel; output, to a display in communication with the processor circuit, a screen display comprising: the image of the blood vessel; a first plaque burden value corresponding to a distal end of the region; a second plaque burden value corresponding to a proximal end of the region; and the plaque burden indicator.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging and x-ray system, according to aspects of the present disclosure.

FIG. 2 is a diagrammatic top view of an ultrasound imaging assembly in a flat configuration, according to aspects of the present disclosure.

FIG. 3 is a diagrammatic perspective view of the ultrasound imaging assembly shown in FIG. 2 in a rolled configuration around a support member, according to aspects of the present disclosure.

FIG. 4 is a diagrammatic cross-sectional side view of the ultrasound imaging assembly shown in FIG. 3, according to aspects of the present disclosure.

FIG. 5 is a schematic diagram of a processor circuit, according to aspects of the present disclosure.

FIG. 6 is a diagrammatic view of an intravascular image, according to aspects of the present disclosure.

FIG. 7 is a diagrammatic view of a relationship between x-ray fluoroscopy images, intravascular ultrasound images, and a path defined by the motion of an intravascular device, according to aspects of the present disclosure.

FIG. 8 is a diagrammatic view of a longitudinal view of an imaged vessel and a plaque burden indication, according to aspects of the present disclosure.

FIG. 9 is a diagrammatic view of a plaque burden indication, according to aspects of the present disclosure.

FIG. 10 is a diagrammatic view of an extraluminal image including a plaque burden indication, according to aspects of the present disclosure.

FIG. 11 is a diagrammatic view of an extraluminal image including a plaque burden indication, according to aspects of the present disclosure.

FIG. 12 is a diagrammatic view of an extraluminal image including a plaque burden indication, according to aspects of the present disclosure.

FIG. 13 is a flow diagram of a method of generating a plaque burden indication and a longitudinal view of a vessel, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

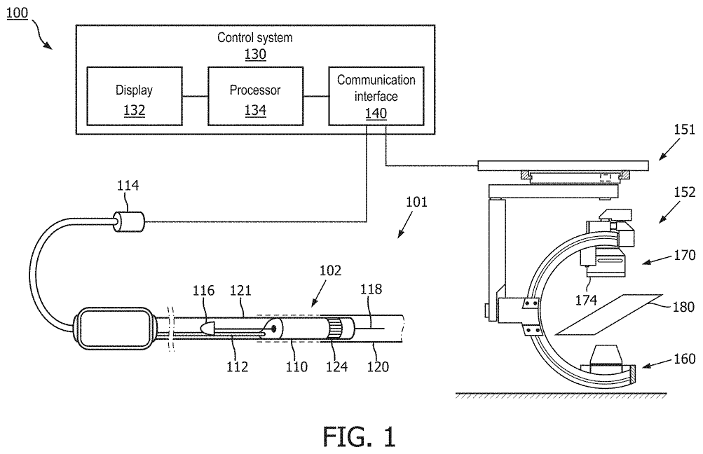

FIG. 1 is a schematic diagram of an intraluminal imaging and x-ray system 100, according to aspects of the present disclosure. In some embodiments, the intraluminal imaging and x-ray system 100 may include two separate systems or be a combination of two systems: an intraluminal sensing system 101 and an extraluminal imaging system 151. The intraluminal sensing system 101 obtains medical data about a patient's body while the intraluminal device 102 is positioned inside the patient's body. For example, the intraluminal sensing system 101 can control the intraluminal device 102 to obtain intraluminal images of the inside of the patient's body while the intraluminal device 102 is inside the patient's body. The extraluminal imaging system 151 obtains medical data about the patient's body while the extraluminal imaging device 152 is positioned outside the patient's body. For example, the extraluminal imaging system 151 can control extraluminal imaging device 152 to obtain extraluminal images of the inside of the patient's body while the extraluminal imaging device 152 is outside the patient's body.

The intraluminal imaging system 101 may be in communication with the extraluminal imaging system 151 through any suitable components. Such communication may be established through a wired cable, through a wireless signal, or by any other means. In addition, the intraluminal imaging system 101 may be in continuous communication with the x-ray system 151 or may be in intermittent communication. For example, the two systems may be brought into temporary communication via a wired cable, or brought into communication via a wireless communication, or through any other suitable means at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging system 101. In some embodiments, and as shown in FIG. 1, the intraluminal imaging system 101 and the x-ray imaging system 151 may be in communication with the same control system 130. In this embodiment, both systems may be in communication with the same display 132, processor 134, and communication interface 140 shown as well as in communication with any other components implemented within the control system 130.

In some embodiments, the system 100 may not include a control system 130 in communication with the intraluminal imaging system 101 and the x-ray imaging system 151. Instead, the system 100 may include two separate control systems. For example, one control system may be in communication with or be a part of the intraluminal imaging system 101 and an additional separate control system may be in communication with or be a part of the x-ray imaging system 151. In this embodiment, the separate control systems of both the intraluminal imaging system 101 and the x-ray imaging system 151 may be similar to the control system 130. For example, each control system may include various components or systems such as a communication interface, processor, and/or a display. In this embodiment, the control system of the intraluminal imaging system 101 may perform any or all of the coregistration steps described in the present disclosure. Alternatively, the control system of the x-ray imaging system 151 may perform the coregistration steps described.

The intraluminal imaging system 101 can be an ultrasound imaging system. In some instances, the intraluminal imaging system 101 can be an intravascular ultrasound (IVUS) imaging system. The intraluminal imaging system 101 may include an intraluminal imaging device 102, such as a catheter, guide wire, or guide catheter, in communication with the control system 130. The control system 130 may include a display 132, a processor 134, and a communication interface 140 among other components. The intraluminal imaging device 102 can be an ultrasound imaging device. In some instances, the device 102 can be an IVUS imaging device, such as a solid-state IVUS device.

At a high level, the IVUS device 102 emits ultrasonic energy from a transducer array 124 included in a scanner assembly, also referred to as an IVUS imaging assembly, mounted near a distal end of the catheter device. The ultrasonic energy is reflected by tissue structures in the surrounding medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The communication interface 140 transfers the received echo signals to the processor 134 of the control system 130 where the ultrasound image (including flow information in some embodiments) is reconstructed and displayed on the display 132. The control system 130, including the processor 134, can be operable to facilitate the features of the IVUS imaging system 101 described herein. For example, the processor 134 can execute computer readable instructions stored on the non-transitory tangible computer readable medium.

The communication interface 140 facilitates communication of signals between the control system 130 and the scanner assembly 110 included in the IVUS device 102. This communication includes the steps of: (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the communication interface 140 performs preliminary processing of the echo data prior to relaying the data to the processor 134. In examples of such embodiments, the communication interface 140 performs amplification, filtering, and/or aggregating of the data. In an embodiment, the communication interface 140 also supplies high- and low-voltage DC power to support operation of the device 102 including circuitry within the scanner assembly 110.

The processor 134 receives the echo data from the scanner assembly 110 by way of the communication interface 140 and processes the data to reconstruct an image of the tissue structures in the medium surrounding the scanner assembly 110. The processor 134 outputs image data such that an image of the lumen 120, such as a cross-sectional image of the vessel 120, is displayed on the display 132. The lumen 120 may represent fluid filled or surrounded structures, both natural and man-made. The lumen 120 may be within a body of a patient. The lumen 120 may be a blood vessel, such as an artery or a vein of a patient's vascular system, including cardiac vasculature, peripheral vasculature, neural vasculature, renal vasculature, and/or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter, Visions PV .014P RX catheter, Visions PV .018 catheter, Visions PV .035, and Pioneer Plus catheter, each of which are available from Koninklijke Philips N. V, and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43AWG gauge wires can be used.

The transmission line bundle 112 terminates in a patient interface module (PIM) connector 114 at a proximal end of the device 102. The PIM connector 114 electrically couples the transmission line bundle 112 to the communication interface 140 and physically couples the IVUS device 102 to the communication interface 140. In some embodiments, the communication interface 140 may be a PIM. In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end to direct the device 102 through the vessel 120.

In some embodiments, the intraluminal imaging device 102 may acquire intravascular images of any suitable imaging modality, including optical coherence tomography (OCT) and intravascular photoacoustic (IVPA).

In some embodiments, the intraluminal device 102 is a pressure sensing device (e.g., pressure-sensing guidewire) that obtains intraluminal (e.g., intravascular) pressure data, and the intraluminal system 101 is an intravascular pressure sensing system that determines pressure ratios based on the pressure data, such as fractional flow reserve (FFR), instantaneous wave-free ratio (iFR), and/or other suitable ratio between distal pressure and proximal/aortic pressure (Pd/Pa). In some embodiments, the intraluminal device 102 is a flow sensing device (e.g., flow-sensing guidewire) that obtains intraluminal (e.g., intravascular) flow data, and the intraluminal system 101 is an intravascular flow sensing system that determines flow-related values based on the pressure data, such as coronary flow reserve (CFR), flow velocity, flow volume, etc.

The x-ray imaging system 151 may include an x-ray imaging apparatus or device 152 configured to perform x-ray imaging, angiography, fluoroscopy, radiography, venography, among other imaging techniques. The x-ray imaging system 151 can generate a single x-ray image (e.g., an angiogram or venogram) or multiple (e.g., two or more) x-ray images (e.g., a video and/or fluoroscopic image stream) based on x-ray image data collected by the x-ray device 152. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 152 may also be in communication with the control system 130. In some embodiments, the x-ray system 151 may include a digital radiography device or any other suitable device.

The x-ray device 152 as shown in FIG. 1 includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 374 may also include a protective screen to shield circuitry or components within the detector 370 from the surrounding environment. In some embodiments, the x-ray detector 170 may include a flat panel detector (FPD). The detector 170 may be an indirect conversion FPD or a direct conversion FPD. The detector 170 may also include charge-coupled devices (CCDs). The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to obtain x-ray images without contrast. In some embodiments, the x-ray imaging system 151 may be configured to obtain x-ray images with contrast (e.g., angiogram or venogram). In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

In some embodiments, the extraluminal imaging system 151 could be any suitable extraluminal imaging device, such as computed tomography (CT) or magnetic resonance imaging (MRI).

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all the organs, fluids, or other structures or parts of an anatomy previously mentioned. In addition to natural structures, the x-ray device 152 may be used to examine man-made structures such as any of the previously mentioned structures.

The processor 134 may be configured to receive an x-ray image that was stored by the x-ray imaging device 152 during a clinical procedure. The images may be further enhanced by other information such as patient history, patient record, IVUS imaging, pre-operative ultrasound imaging, pre-operative CT, or any other suitable data.

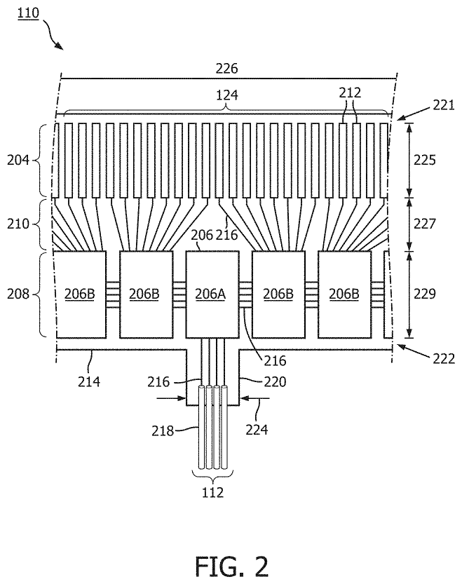

FIG. 2 is a diagrammatic top view of a portion of a flexible assembly 110, according to aspects of the present disclosure. The flexible assembly 110 includes a transducer array 124 formed in a transducer region 204 and transducer control logic dies 206 (including dies 206A and 206B) formed in a control region 208, with a transition region 210 disposed therebetween. The transducer array 124 includes an array of ultrasound transducer elements 212. The transducer control logic dies 206 are mounted on a flexible substrate 214 into which the transducer elements 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

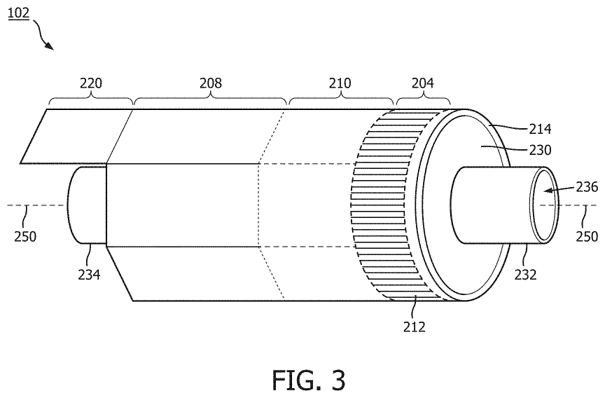

The flexible substrate 214, on which the transducer control logic dies 206 and the transducer elements 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 with some particular embodiments being between 5 μm and 25.1 e.g., 6 μm

The set of transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 112, between a processing system, e.g., processing system 106, and the flexible assembly 110. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a plurality of transducer elements 512 positioned on a transducer element 212 to emit an ultrasonic signal and selects a transducer element 212 to receive an echo. In the depicted embodiment, the master controller 206A does not directly control any transducer elements 212. In other embodiments, the master controller 206A drives the same number of transducer elements 212 as the slave controllers 206B or drives a reduced set of transducer elements 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducer elements 212, in an embodiment, the flexible substrate 214 includes conductive traces 216 formed in the film layer that carry signals between the control logic dies 206 and the transducer elements 212. In particular, the conductive traces 216 providing communication between the control logic dies 206 and the transducer elements 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace or pad.

The flexible substrate 214 can include a conductor interface 220 in some embodiments. The conductor interface 220 can be in a location of the flexible substrate 214 where the conductors 218 of the cable 112 are coupled to the flexible substrate 214. For example, the bare conductors of the cable 112 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the scanner assembly 110 in a rolled configuration. In some instances, the flexible substrate 214 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

Depending on the application and embodiment of the presently disclosed invention, transducer elements 212 may be piezoelectric transducers, single crystal transducer, or PZT (lead zirconate titanate) transducers. In other embodiments, the transducer elements of transducer array 124 may be flexural transducers, piezoelectric micromachined ultrasonic transducers (PMUTs), capacitive micromachined ultrasonic transducers (CMUTs), or any other suitable type of transducer element. In such embodiments, transducer elements 212 may comprise an elongate semiconductor material or other suitable material that allows micromachining or similar methods of disposing extremely small elements or circuitry on a substrate.

In some embodiments, the transducer elements 212 and the controllers 206 can be positioned in an annular configuration, such as a circular configuration or in a polygon configuration, around a longitudinal axis 250 of a support member 230. It is understood that the longitudinal axis 250 of the support member 230 may also be referred to as the longitudinal axis of the scanner assembly 110, the flexible elongate member 121, or the device 102. For example, a cross-sectional profile of the imaging assembly 110 at the transducer elements 212 and/or the controllers 206 can be a circle or a polygon. Any suitable annular polygon shape can be implemented, such as one based on the number of controllers or transducers, flexibility of the controllers or transducers, etc. Some examples may include a pentagon, hexagon, heptagon, octagon, nonagon, decagon, etc. In some examples, the transducer controllers 206 may be used for controlling the ultrasound transducers 512 of transducer elements 212 to obtain imaging data associated with the vessel 120.

The support member 230 can be referenced as a unibody in some instances. The support member 230 can be composed of a metallic material, such as stainless steel, or a non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. In some embodiments, support member 230 may be composed of 303 stainless steel. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process or a micro injection molding process.

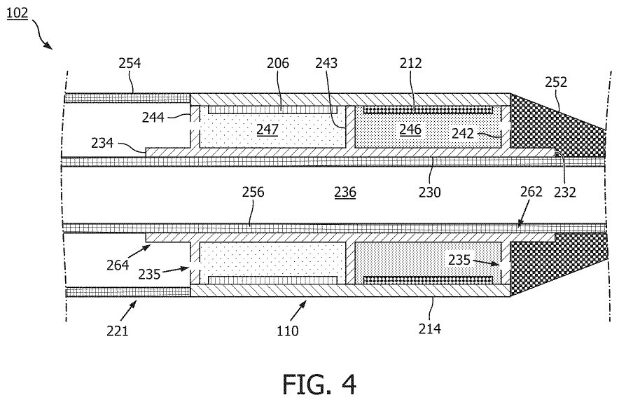

Referring now to FIG. 4, shown therein is a diagrammatic cross-sectional side view of a distal portion of the intraluminal imaging device 102, including the flexible substrate 214 and the support member 230, according to aspects of the present disclosure. The lumen 236 may be connected with the entry/exit port 116 and is sized and shaped to receive the guide wire 118 (FIG. 1). In some embodiments, the support member 230 may be integrally formed as a unitary structure, while in other embodiments the support member 230 may be formed of different components, such as a ferrule and stands 242, 243, and 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 243, and 244 that extend vertically are provided at the distal, central, and proximal portions respectively, of the support member 230. The stands 242, 243, and 244 elevate and support the distal, central, and proximal portions of the flexible substrate 214. In that regard, portions of the flexible substrate 214, such as the transducer portion 204 (or transducer region 204), can be spaced from a central body portion of the support member 230 extending between the stands 242, 243, and 244. The stands 242, 243, 244 can have the same outer diameter or different outer diameters. For example, the distal stand 242 can have a larger or smaller outer diameter than the central stand 243 and/or proximal stand 244 and can also have special features for rotational alignment as well as control chip placement and connection.

To improve acoustic performance, the cavity between the transducer array 212 and the surface of the support member 230 may be filled with an acoustic backing material 246. The liquid backing material 246 can be introduced between the flexible substrate 214 and the support member 230 via passageway 235 in the stand 242, or through additional recesses as will be discussed in more detail hereafter. The backing material 246 may serve to attenuate ultrasound energy emitted by the transducer array 212 that propagates in the undesired, inward direction.

The cavity between the circuit controller chips 206 and the surface of the support member 230 may be filled with an underfill material 247. The underfill material 247 may be an adhesive material (e.g. an epoxy) which provides structural support for the circuit controller chips 206 and/or the flexible substrate 214. The underfill 247 may additionally be any suitable material.

In some embodiments, the central body portion of the support member can include recesses allowing fluid communication between the lumen of the unibody and the cavities between the flexible substrate 214 and the support member 230. Acoustic backing material 246 and/or underfill material 247 can be introduced via the cavities (during an assembly process, prior to the inner member 256 extending through the lumen of the unibody. In some embodiments, suction can be applied via the passageways 235 of one of the stands 242, 244, or to any other suitable recess while the liquid backing material 246 is fed between the flexible substrate 214 and the support member 230 via the passageways 235 of the other of the stands 242, 244, or any other suitable recess. The backing material can be cured to allow it to solidify and set. In various embodiments, the support member 230 includes more than three stands 242, 243, and 244, only one or two of the stands 242, 243, 244, or none of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions of the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can comprise a flexible elongate member. The proximal inner member 256 can be received within a proximal flange 234. The proximal outer member 254 abuts and is in contact with the proximal end of flexible substrate 214. A distal tip member 252 is coupled to the distal portion 262 of the support member 230. For example, the distal member 252 is positioned around the distal flange 232. The tip member 252 can abut and be in contact with the distal end of flexible substrate 214 and the stand 242. In other embodiments, the proximal end of the tip member 252 may be received within the distal end of the flexible substrate 214 in its rolled configuration. In some embodiments there may be a gap between the flexible substrate 214 and the tip member 252. The distal member 252 can be the distal-most component of the intraluminal imaging device 102. The distal tip member 252 may be a flexible, polymeric component that defines the distal-most end of the imaging device 102. The distal tip member 252 may additionally define a lumen in communication with the lumen 236 defined by support member 230. The guide wire 118 may extend through lumen 236 as well as the lumen defined by the tip member 252.

One or more adhesives can be disposed between various components at the distal portion of the intraluminal imaging device 102. For example, one or more of the flexible substrate 214, the support member 230, the distal member 252, the proximal inner member 256, the transducer array 212, and/or the proximal outer member 254 can be coupled to one another via an adhesive. Stated differently, the adhesive can be in contact with e.g. the transducer array 212, the flexible substrate 214, the support member 230, the distal member 252, the proximal inner member 256, and/or the proximal outer member 254, among other components.

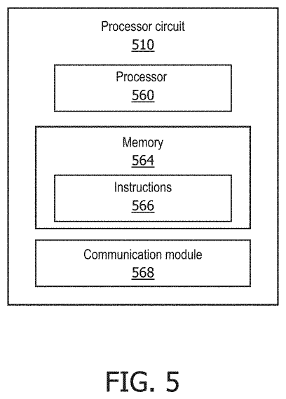

FIG. 5 is a schematic diagram of a processor circuit, according to aspects of the present disclosure. The processor circuit 510 may be implemented in the control system 130 of FIG. 1, the intraluminal imaging system 101, and/or the x-ray imaging system 151, or any other suitable location. In an example, the processor circuit 510 may be in communication with intraluminal imaging device 102, the x-ray imaging device 152, the display 132 within the system 100. The processor circuit 510 may include the processor 134 and/or the communication interface 140 (FIG. 1). One or more processor circuits 510 are configured to execute the operations described herein. As shown, the processor circuit 510 may include a processor 560, a memory 564, and a communication module 568. These elements may be in direct or indirect communication with each other, for example via one or more buses.

The processor 560 may include a CPU, a GPU, a DSP, an application-specific integrated circuit (ASIC), a controller, an FPGA, another hardware device, a firmware device, or any combination thereof configured to perform the operations described herein. The processor 560 may also be implemented as a combination of computing devices, e.g., a combination of a DSP and a microprocessor, a plurality of microprocessors, one or more microprocessors in conjunction with a DSP core, or any other such configuration.

The memory 564 may include a cache memory (e.g., a cache memory of the processor 560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 564 includes a non-transitory computer-readable medium. The memory 564 may store instructions 566. The instructions 566 may include instructions that, when executed by the processor 560, cause the processor 560 to perform the operations described herein with reference to the probe 110 and/or the host 130 (FIG. 1). Instructions 566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 568 can include any electronic circuitry and/or logic circuitry to facilitate direct or indirect communication of data between the processor circuit 510, the probe 110, and/or the display 132 and/or display 132. In that regard, the communication module 568 can be an input/output (I/O) device. In some instances, the communication module 568 facilitates direct or indirect communication between various elements of the processor circuit 510 and/or the probe 110 (FIG. 1) and/or the host 130 (FIG. 1).

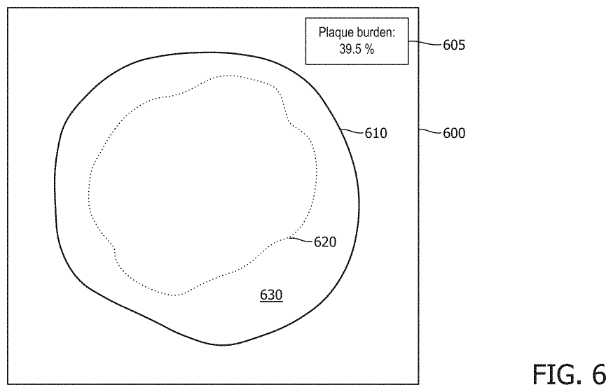

FIG. 6 is a diagrammatic view of an intravascular image 600, according to aspects of the present disclosure. The image 600 may be received by the intraluminal imaging system 101 from, for example, the device 102. In one example, the intraluminal image 600 may be an IVUS image 600.

In some embodiments, the processor circuit 510 may analyze each IVUS image acquired with the intraluminal imaging system 101 and may identify a vessel wall 610 and a lumen boundary 620 within each image. The circuit 510 may identify these structures by any suitable technique, process, or algorithm. For example, the circuit 510 may automatically identify a vessel wall 610 and/or a lumen boundary 620 of an IVUS image, such as the image 600. Examples of border detection, image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

In some embodiments, the processor circuit 510 may use various image processing techniques to identify the vessel wall 610 and the lumen boundary 620 within each IVUS image frame. For example, the processor circuit 510 may use an edge-detection technique. In other embodiments, the circuit 510 may employ other image processing techniques such as pixel-by-pixel analysis to determine transitions between light pixels and dark pixels, filtering, or any other suitable techniques.

In some embodiments, the vessel wall 610 may be associated with an external elastic lamina (EEL). For example, the processor circuit 510 may be configured to identify the EEL of a vessel wall for each given IVUS image. In some embodiments, the vessel wall 610 may be associated with any other part of the vessel. For example, the vessel wall 610 identified by the processor circuit 510 may be associated with an endothelium layer, a subendothelial layer, an internal elastic lamina, a tunica media layer, a tunica externa layer, or any other structure.

After the circuit 510 has identified a vessel wall 610 and a lumen boundary 620 within each IVUS image 600, the circuit 510 may determine a plaque burden 605 associated with that image. A plaque burden may include a numerical representation of the amount of plaque present in a particular IVUS image. In one example, a plaque burden may convey a difference between a cross-sectional area of a vessel and a cross-sectional area of a lumen. In one example, a plaque burden may include a percentage. In one embodiment, a plaque burden may be calculated by determining the cross-sectional area of the vessel wall 610. The cross-sectional area of the lumen 620 may also be calculated. The cross-sectional area of the lumen 620 may then be subtracted from the cross-sectional area of the vessel 610. This difference then relates to the cross-sectional area of material which blocks blood flow within the vessel. In some embodiments, this relates to the cross-sectional area of plaque shown in the IVUS image 600. This cross-sectional area may then be divided by the cross-sectional area of the vessel wall 610 to yield a percentage of the cross-sectional area of the vessel wall 610 which corresponds to plaque. This percentage may be displayed to a user of the system as a plaque burden 605. The plaque burden 605 for each IVUS image received (e.g., image 600) may be stored in a memory in communication with the processor circuit 510 (e.g., the memory 564 of FIG. 5). In some embodiments, the plaque burden 605 may be displayed overlaid on the IVUS image 600, adjacent to or proximate to the IVUS image 600, or displayed in any other way. In some embodiments, the plaque burden 605 may not be displayed. In some embodiments, a visual characteristic of the image 600 or of representations of the image 600 may be altered to convey the plaque burden 605 associated with the image 600. For example, various colors, shapes, symbols, patterns, transparencies, or any suitable visual characteristics may be used to show the value of the plaque burden of the image 600.

In some embodiments, a region 630 between the vessel wall 610 and the lumen boundary 620 may be identified or highlighted within the image 600. For example, the region 630 may correspond to a cross-sectional view of material of a constriction of the vessel at this location. In some cases, the region 630 may correspond to plaque. For the purposes of the present disclosure, plaque may be defined as any material which obstructs blood flow within a vessel. For the purposes of this disclosure, a cross-sectional view of a body lumen may include a region corresponding to the lumen, which may be a region which allows blood flow, and a region corresponding to plaque, which may be a region which does not allow blood flow. Plaque may include any suitable type of material including calcium, fibrous tissues, fibro-fatty tissues, lipids, complex carbohydrates, necrotic core, dead blood cells, or any other material.

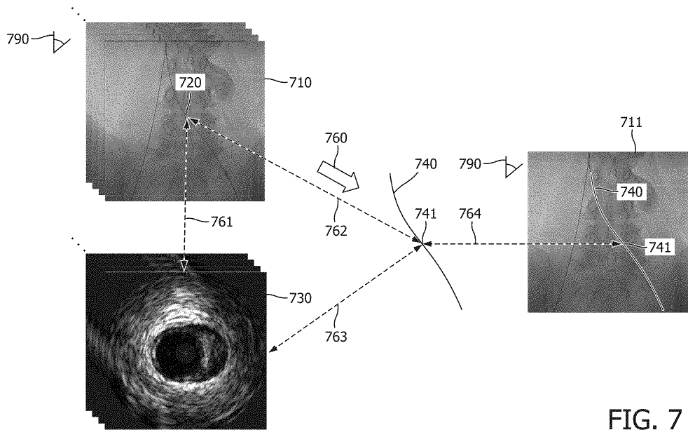

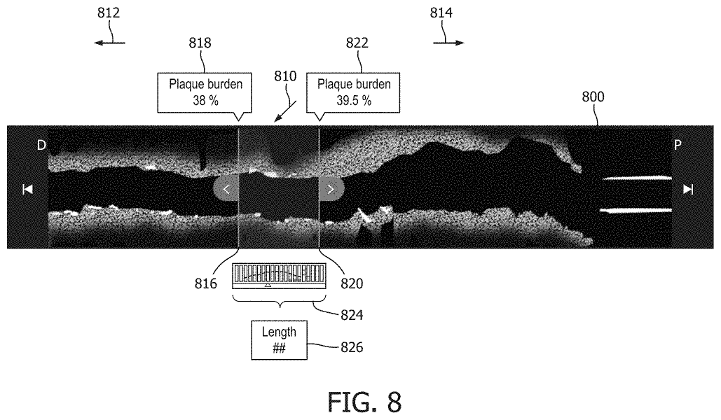

The plaque burden value 605 may be stored in a memory in conjunction with the image 600 and/or the intravascular data associated with the image 600. As will be explained in more detail with reference to FIG. 7, any of these data may be associated with locations along the imaged vessel by coregistration. As described, the processor circuit 510 may auto-segment IVUS images to identify an intraluminal border and an EEL. Using the output of the auto segmentation of the intraluminal border and EEL, a plaque burden can be quantified for every IVUS pullback image. In addition, the processor circuit 510 may be configured to automatically characterize the plaque on input. The processor circuit 510 may then output a visual display of plaque burden and type, superimposed on the angiogram or the longitudinal view of the vessel (e.g., an ILD) and geographically co-located with the diseased segment, as will be described with more detail with reference to FIGS. 8-12.

FIG. 7 is a diagrammatic view of a relationship between x-ray fluoroscopy images 710, intravascular data 730, and a path 740 defined by the motion of an intravascular device, according to aspects of the present disclosure. FIG. 7 describes a method of coregistering intravascular data 730 including intravascular images with corresponding locations on one or more fluoroscopy images 710 of the same region of a patient's anatomy. In particular, and as will be described, at one step of the method, an operator performs an angiogram imaging procedure. The operator may also perform an IVUS pullback procedure simultaneously.

The patient anatomy may be imaged with an x-ray device while a physician performs a pullback with an intravascular device 720, e.g., while the intravascular device 720 moves through a blood vessel of the anatomy. The intravascular device may be substantially similar to the intravascular device 102 described with reference to FIG. 1. The x-ray device used to obtain the fluoroscopy images 710 may be substantially similar to the x-ray device 152 of FIG. 1. In some embodiments, the fluoroscopy images 710 may be obtained while no contrast agent is present within the patient vasculature. Such an embodiment is shown by the fluoroscopy images 710 in FIG. 7. The radiopaque portion of the intravascular device 720 is visible within the fluoroscopy image 710. The fluoroscopy images 710 may correspond to a continuous image stream of fluoroscopy images and may be obtained as the patient anatomy is exposed to a reduced dose of x-radiation. It is noted that the fluoroscopy images 710 may be acquired with the x-ray source 160 and the x-ray detector 170 positioned at any suitable angle in relation to the patient anatomy. This angle is shown by angle 790.

The intravascular device 720 may be any suitable intravascular device. As the intravascular device 720 moves through the patient vasculature, the x-ray imaging system may acquire multiple fluoroscopy images 710 showing the radiopaque portion of the intravascular device 720. In this way, each fluoroscopy image 710 shown in FIG. 7 may depict the intravascular device 720 positioned at a different location such that a processor circuit may track the position of the intravascular device 720 over time.

As the intravascular device 720 is pulled through the patient vasculature, it may acquire intravascular data 730. In an example, the intravascular data 730 shown in FIG. 7 may be IVUS images. However, the intravascular data may be any suitable data, including IVUS images, FFR data, iFR data, OCT images, intravascular photoacoustic (IVPA) images, or any other measurements or metrics relating to blood pressure, blood flow, lumen structure, or other physiological data acquired during a pullback of an intravascular device. As described with reference to FIG. 6, the intravascular data 730 associated with a particular IVUS image may include, among other data, raw intravascular data acquired by the intraluminal imaging device 102, an IVUS image, location data corresponding to a vessel wall, location data corresponding to a lumen boundary, a cross-sectional area defined by a vessel wall, a cross-sectional area defined by a lumen boundary, and a plaque burden value. As described with reference to FIG. 7, any of these data may be coregistered to locations along a pathway and a location within an image 711.

As the physician pulls the intravascular device 720 through the patient vasculature, each intravascular data point 730 acquired by the intravascular device 720 may be associated with a position within the patient anatomy in the fluoroscopy images 710, as indicated by the arrow 761. For example, the first IVUS image 730 shown in FIG. 7 may be associated with the first fluoroscopy image 710. The first IVUS image 730 may be an image acquired by the intravascular device 720 at a position within the vasculature, as depicted in the first fluoroscopy image 710 as shown by the intravascular device 720 within the image 710. Similarly, an additional IVUS image 730 may be associated with an additional fluoroscopy image 710 showing the intravascular device 720 at a new location within the image 710, and so on. The processor circuit may determine the locations of the intravascular device 720 within each acquired x-ray image 710 by any suitable method. For example, the processor circuit may perform various image processing techniques, such as edge identification of the radiopaque marker, pixel-by-pixel analysis to determine transition between light pixels and dark pixels, filtering, or any other suitable techniques to determine the location of the imaging device 720. In some embodiments, the processor circuit may use various artificial intelligence methods including deep learning techniques such as neural networks or any other suitable techniques to identify the locations of the imaging device 720 within the x-ray images 710.

Any suitable number of IVUS images or other intravascular data points 730 may be acquired during an intravascular device pullback and any suitable number of fluoroscopy images 710 may be obtained. In some embodiments, there may be a one-to-one ratio of fluoroscopy images 710 and intravascular data 730. In other embodiments, there may be differing numbers of fluoroscopy images 710 and/or intravascular data 730. The process of co-registering the intravascular data 730 with one or more x-ray images may include some features similar to those described in U.S. Pat. No. 7,930,014, titled, “VASCULAR IMAGE CO-REGISTRATION,” and filed Jan. 11, 2006, which is hereby incorporated by reference in its entirety. The co-registration process may also include some features similar to those described in U.S. Pat. Nos. 8,290,228, 8,463,007, 8,670,603, 8,693,756, 8,781,193, 8,855,744, and 10,076,301, all of which are also hereby incorporated by reference in their entirety.

The system 100 may additionally generate a fluoroscopy-based 2D pathway 740 defined by the positions of the intravascular device 720 within the x-ray fluoroscopy images 710. The different positions of the intravascular device 720 during pullback, as shown in the fluoroscopy images 710, may define a two-dimensional pathway 740, as shown by the arrow 760. The fluoroscopy-based 2D pathway 740 reflects the path of one or more radiopaque portions of the intravascular device 720 as it moved through the patient vasculature as observed from the angle 790 by the x-ray imaging device 152. The fluoroscopy-based 2D pathway 740 defines the path as measured by the x-ray device which acquired the fluoroscopy images 710, and therefore shows the path from the same angle 790 at which the fluoroscopy images were acquired. Stated differently, the 2D pathway 740 describes the projection of the 3D path followed by the device onto the imaging plane at the imaging angle 790. In some embodiments, the pathway 740 may be determined by an average of the detected locations of the intravascular device 720 in the fluoroscopy images 710. For example, the pathway 740 may not coincide exactly with the guidewire in any fluoroscopy image 710 selected for presentation.

As shown by the arrow 762, because the two-dimensional path 740 is generated based on the fluoroscopy images 710, each position along the two-dimensional path 740 may be associated with one or more fluoroscopy images 710. As an example, at a location 741 along the path 740, the first fluoroscopy image 710 may depict the intravascular device 720 at that same position 741. In addition, because a correspondence was also established between the fluoroscopy images 710 and the intravascular data 730 as shown by the arrow 761, intravascular data 730, such as the first IVUS image shown, may also be associated with the location 741 along the path 740 as shown by the arrow 763.

Finally, the path 740 generated based on the locations of the intravascular device 720 within the fluoroscopy images 710 may be overlaid onto any suitable fluoroscopy image 711 (e.g., one of the fluoroscopic images 710 in the fluoroscopic image stream). In this way, any location along the path 740 displayed on the fluoroscopy image 711 may be associated with IVUS data such as an IVUS image 730, as shown by the arrow 764. For example, the first IVUS image 730 shown in FIG. 7 may be acquired simultaneously with the first fluoroscopy image 710 shown and the two may be associated with each other as shown by the arrow 761. The fluoroscopy image 710 may be analyzed to determine the location of the intravascular device 720 along the path 740, as shown by the arrow 762, thus associating the IVUS image 730 with the location 741 along the path 740 as shown by the arrow 763. Finally, the IVUS image 730 may be associated with the location within the fluoroscopy image 711 at which it was acquired by overlaying the path 740 with associated data on the fluoroscopy image 711. The pathway 740 itself may or may not be displayed on the image 711.

In the illustrated embodiment of FIG. 7, the co-registered IVUS images are associated with one of the fluoroscopic images obtained without contrast such that that the position at which the IVUS images are obtained is known relative to locations along the guidewire. In other embodiments, the co-registered IVUS images are associated with an x-ray image obtained with contrast (in which the vessel is visible) such that that the position at which the IVUS images are obtained is known relative to locations along the vessel.

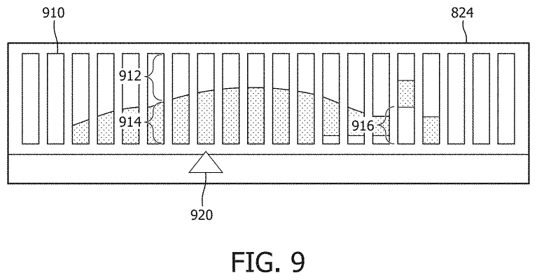

FIG. 8 is a diagrammatic view of a longitudinal view 800 of an imaged vessel and a plaque burden indication 824, according to aspects of the present disclosure. As shown in FIG. 8, an automated IVUS derived plaque burden indication is disclosed. FIG. 8 shows one example of longitudinal intraluminal image 800. The longitudinal image 800 can be referred to as in-line digital (ILD) display or image longitudinal display (ILD). The IVUS images acquired during an intravascular ultrasound imaging procedure, such as during an IVUS pullback, may be used to create the ILD 800. In that regard, an IVUS image is a tomographic or radial cross-sectional view of the blood vessel. The ILD 800 provides a longitudinal cross-sectional view of the blood vessel. The ILD 800 can be a stack of the IVUS images acquired at various positions along the vessel, such that the longitudinal view of the ILD 800 is perpendicular to the radial cross-sectional view of the IVUS images. In such an embodiment, the ILD 800 may show the length of the vessel, whereas an individual IVUS image is a single radial cross-sectional image at a given location along the length. In another embodiment, the ILD 800 may be a stack of the IVUS images acquired overtime during the imaging procedure and the length of the ILD 800 may represent time or duration of the imaging procedure. The ILD 800 may be generated and displayed in real time or near real time during the pullback procedure. As each additional IVUS image is acquired, it may be added to the ILD 800. For example, at a point in time during the pullback procedure, the ILD 800 shown in FIG. 9 may be partially complete. In some embodiments, the processor circuit may generate an illustration of a longitudinal view of the vessel being imaged based on the received IVUS images. For example, rather than displaying actual vessel image data as the ILD 800 does, the illustration may be a stylized version of the vessel, with e.g., continuous lines showing the lumen border and vessel border.

As previously described with reference to FIG. 6 and FIG. 7, each IVUS image received during a pullback procedure may be analyzed to determine a vessel wall and lumen boundary. These calculations may be used to determine a plaque burden value. In that regard, each IVUS image of the IVUS images received by the processor circuit 510 may be associated with at least one plaque burden value. As the ILD 800 is generated based on these IVUS images, each location along the ILD 800 may be associated with at least one IVUS image and at least one plaque burden value. As a result, the processor circuit 510 may be configured to display a plaque burden value or score associated with a location along the ILD 800 in response to a user input selecting a location along the ILD 800.

In some embodiments, the processor circuit 510 (FIG. 5) may automatically recommend a stent type and placement to a user of the system. In one embodiment, the processor circuit 510 may compare lumen boundary diameters or cross-sectional areas of all the received IVUS images. In one embodiment, the processor circuit 510 may compare plaque burden values associated with all the received IVUS images. Based on these comparisons, the processor circuit may identify regions of interest along the imaged vessel. For example, the processor circuit 510 may select a region, such as one or more IVUS images, corresponding to a minimum lumen diameter or minimum lumen area of the imaged vessel. The processor circuit may select multiple IVUS images corresponding to local minima of lumen diameter and/or lumen area. In some embodiments, the processor circuit may select one or more IVUS images corresponding to a maximum plaque burden of the imaged vessel or local maxima of plaque burden. In some embodiments, the processor circuit 510 may select regions of interest based on any or all of a minimum lumen diameter, a minimum lumen area, or a plaque burden.

In some embodiments, the processor circuit 510 may recommend a location for a stent based on the regions of interest selected. For example, the processor circuit 510 may recommend a stent placement such that a central portion of a recommended stent is to be placed at a region of interest.

In some embodiments, the processor circuit 510 may recommend a location of a stent such that the two ends, the proximal and distal ends, of the recommended stent are positioned at healthy regions of the vessel. For example, the processor circuit 510 may compare the plaque burden value for each IVUS image extending distally and proximally from a region of interest to a threshold plaque burden value. The processor circuit 510 may generate this threshold plaque burden value automatically based on features or measurements of the vessel imaged or features or measurements of other vessels of the same patient, other patients with similar features, diagnoses, or characteristics, or based on any other factors. In some embodiments, the processor circuit 510 may determine the threshold plaque burden value in response to a recommended threshold value, such as one determined by experts in the field, a user input, or a user setting.

One pedagogical example is shown in FIG. 8. In this example, a threshold plaque burden may be determined to be 40%. Using any of the methods or techniques disclosed previously, the processor circuit 510 may have identified a region of interest 810 along the ILD 800. The region 810 may correspond to a lesion, blockage, or constriction of the vessel, or any other observed condition. In this example, a plaque burden calculated for this region of interest 810 may be, for example, 50%. The processor circuit may then analyze the plaque burden values of images both distal and proximal to the location of the region of interest 819. As an example, the processor circuit 510 may analyze each IVUS image in a distal direction 812. The first IVUS image in a distal direction 812 from the region of interest 810 with a corresponding plaque burden value below the threshold value, in this case 40%, may be selected and displayed. In this example, this image is identified and displayed to a user with the indicator 816. An additional indicator 818 may display the plaque burden value associated with the IVUS image shown by the indicator 816. In this example, the indicator 818 may display a plaque burden value of 38%. The indicator 818 advantageously provides a numerical value that communicates to a user that the planned/proposed distal landing zone is within healthy tissue if the indicator 818 displays a plaque burden that is less than the threshold.

Continuing with the foregoing example, the processor circuit 510 may analyze the IVUS images in a proximal direction 814 from the region of interest 810. The first IVUS image in a proximal direction 814 from the region of interest 810 with a corresponding plaque burden value below the threshold value, in this case 40%, may be selected and displayed. In this example, this image is identified and displayed to a user with the indicator 820. An additional indicator 822 may display the plaque burden value associated with the IVUS image shown by the indicator 820. In this example, this indicator 822 may display a plaque burden value of 39.5%. The indicator 820 advantageously provides a numerical value that communicates to a user that the planned/proposed proximal landing zone is within healthy tissue if the indicator 820 displays a plaque burden that is less than the threshold. In some embodiments, the indicators 818 and 822 depicting the plaque burden values at the distal and proximal stent edges may be calculated and displayed automatically. An automated solution may be displayed at cursors. Automated quantification reduces procedure time.

In some embodiments, the region between the indicators 816 and 820 may be identified for a user on the display. For example, this region may be differentiated from other regions of the ILD 800 by highlighting, coloring, shading, by an overlaid pattern, or by any other method. For example, the region between the indicators 816 and 820 may be shaded with a particular color or highlight, may be of a reduced or increased transparency in comparison with other regions of the ILD 800, may include an overlay of any particular color, pattern, or transparency, or differentiated in another way.

In some embodiments, the indicators 816 and 820 and/or the region between these indicators may correspond to a stent placement recommendation. In that regard, the indicator 816 may correspond to a distal landing zone of a recommended stent and the indicator 820 may correspond to a proximal landing zone of a recommended stent. The length between the indicator 816 and the indicator 820 may, therefore, correspond to a length of a recommended stent. In this way, a stent plan may be applied and proximal and distal stent edge markers may be displayed on the screen.

The initial positioning of the distal and proximal markers (e.g., the indicators 816 and 820) may be automatic or manual (i.e., based on a user input). In some embodiments, the system may analyze each IVUS image frame as described with reference to FIG. 6. In this way, the system may find a minimum luminal diameter (MLD) frame. The processor circuit 510 may then predict where the best distal and proximate sites are and display them to a user. These predicted sites for distal and proximal landing zones may correspond to the nearest healthy regions in both a proximal and distal direction from the MLD frame. The processor circuit 510 may then provide a way for the user to edit the location of the proximal and distal sites. The user may then move the proximal and distal sites to a healthy region on either side of the MLD frame and may determine a more suitable placement for the stent.

Referring again to FIG. 8, the ILD 800 may be accompanied by any suitable indicators identifying a length between the indicators 816 and 820. For example, the graphic 826 may be provided. The graphic 826 may identify the region between the indicators 816 and 820 and may display to the user of the system a length measurement between the indicators 816 and 820. The length measurement may be in any suitable unit, such as mm, cm, m, French, or any other unit. In some embodiments, a physician may select a stent for deployment at the region 810 of the vessel in part based on this length measurement 826.

In some embodiments, the processor circuit 510 may be configured to receive additional user inputs after displaying any or all of the elements shown in FIG. 8. For example, the circuit 510 may receive an input from a user moving either of the indicators 816 or 820. The user may generate this input by any suitable input device. For example, this input may include a gesture on a touch screen device, one or more strokes on a keyboard, a selection and/or movement with a mouse, or any other form of input. In response to the user input moving the indicator 816 in a direction 812, for example, the circuit may display the indicator 816 at a new position distal of the original position. In addition, in response to this input, the indicator 818 may also be updated to display a new plaque burden value associated with the new location. A longitudinal plaque burden graphic 824, discussed in detail hereafter, may also be updated. For example, as proximal and distal stent edge markers are moved, plaque burden percentages are updated on the markers, allowing for the physician to determine optimal landing zones for the stent (away from plaque) to improve stenting and patient outcomes.

The longitudinal plaque burden graphic is a graphical representation of numerical values associated with the plaque burden at various locations along the ILD. In some embodiments, the longitudinal plaque burden graphic may be a stacked bar graph or chart, with each segment 910 (FIG. 9) being a stacked bar. Each bar within a segment 910 may represent a percentage of the cross-sectional area of the vessel. In that regard, each bar within a segment 910 may add up to a whole, e.g., 100%. Different colors of the bars of each segment 910 may correspond to different regions within an IVUS image (e.g., calcium area, plaque area, lumen area, etc.). Additional aspects of the longitudinal plaque burden graphic 824 will be described in greater detail with reference to FIG. 9. However, as the indicator 816 is moved (e.g., in a distal direction), the graphic 824 may be updated to be a of a different shape (e.g., a longer shape) and may include additional segments 910 associated with any additional regions of the ILD 800 now included between the new position of the indicator 816 and the indicator 820. The length graphic 826 may also be updated to reflect a new length measurement.

The processor circuit 510 may perform a similar process of updating any relevant metrics in response to a user moving the indicator 820. In particular, a user input may alternatively instruct the processor circuit 510 to move the location of the indicator 820 in either a proximal direction 814 or a distal direction 812. In response to such an input, the position of the indicator 820 may be adjusted, the plaque burden 822 may be recalculated to reflect the new position of the indicator 820, the longitudinal plaque burden graphic 824 may be updated to include additional segments 910 or exclude previously included segments 910, and the length metric 826 may be updated based on the new position of the indicator 820.

FIG. 9 is a diagrammatic view of a plaque burden indication 824, according to aspects of the present disclosure. The longitudinal plaque burden graphic 824 may alternatively be referred to as a plaque burden indication 824. Referring to FIG. 9, an expanded view of the longitudinal plaque burden graphic 824 is shown. A longitudinal plaque burden graphic 824 may convey to a user any number or types of measurements related to received IVUS images in a variety of ways. In one example, the graphic 824 shown may include multiple segments 910. Each segment 910 may correspond to a single IVUS image received or multiple IVUS images. For example, each segment may correspond to 2, 5, 10, 20 or more IVUS images or any number therebetween. Each segment 910 may additionally be visually distinguished from one another to convey various aspects or measurements of the IVUS image(s) to which the segment 910 is associated. As an example, two regions 912 and 914 of a segment 910 are identified within FIG. 9. The regions 912 and 914 may be visually differentiated from one another. For example, the region 912 may be of a different color than the region 914. The regions 912 and 914 may alternatively or additionally be differentiated from one another in any other suitable way, including varying borders, shading, patterns, or by any other visual characteristics. The relative size of the region 912 vs the region 914 may indicate any number of metrics. For example, the size of the region 914 may correspond to the plaque burden value of an IVUS image associated with the segment. In an embodiment in which one segment 910 is associated with multiple IVUS images, the size of the region 914 may correspond to some combination of the plaque burden values of all the associated IVUS images (e.g., an average, median, or other combination). By contrast, the region 912 may correspond to a percentage of the vessel area which is not blocked or does not correspond to an obstruction or plaque. For example, the region 912 may correspond to a percentage value corresponding to the difference between the plaque burden value and a value of 100%.

In some embodiments, additional regions of a segment 910 may be included, such as a region 916. The region 916 may correspond to an additional metric associated with the IVUS images corresponding to the segments 910 and may be further distinguished visually from the regions 912 and 914. The region 916 may be visually distinguished from the regions 912 and 914 in any way, including any of those described with reference to the regions 912 and 914. For example, in some embodiments, regions 916 of segments 910 may correspond to a percentage of a vessel cross-sectional area of calcium deposits. In such an embodiment, the region 916 may be a subset of the region 914. It is noted, however, that the region 916, as well as the regions 912 and 914, may correspond to any metric of the associated IVUS images.

In some embodiments, the longitudinal plaque burden graphic 824 may include an indicator 920. The indicator 920 may correspond to the location of a selected IVUS image, selected position on a corresponding extraluminal image, or a location on a corresponding image-based ILD, such as the ILD 800, or another ILD, including a measurement-based ILD or stylized ILD. In some embodiments, the indicator 920 may correspond to the location of the region of interest 810 (FIG. 8). In some embodiments, multiple indicators 920 may be displayed in connection with the longitudinal plaque burden graphic 824.

The longitudinal plaque burden graphic 824 may quickly and efficiently convey to the user of the system 100 a view of the plaque burden of the region between the indicators 816 and 820 (FIG. 8). The longitudinal plaque burden graphic 824 may also advantageously provide a user with an efficient view of whether the distal and proximal landing zones of a planned stent are within healthy regions of the vessel. For example, a user may ensure that the ends of a stent are within healthy regions of the vessel if each outermost segments 910 (e.g., the distal most segment 910 and the proximal most segment 910) both include a single region 912, or a region corresponding to a lumen cross-sectional area denoting that no plaque is present at the associated locations along the vessel. In some embodiments, a user may ensure that the regions 912 of the outermost segments 910 are a great majority of the segments 910, e.g., above a threshold. For example, some amount of plaque may be present within a segment 910 and still correspond to a healthy region of the vessel. This determination may be made by comparing the plaque burden percentages of regions 912, 914, and/or 916 with one or more threshold values. In some cases, a user may ensure that additional segments 910 adjacent to the outermost segments 910 also include only regions 912 or primarily regions 912 denoting a healthy region of the vessel. A plaque value quantification may include a comparison of lumen and vessel areas. As a user moves the handles, the processor circuit may automatically update the plaque burden on either side This allows a user to make sure that the landing zones are in healthy regions.

In some embodiments, the processor circuit 510 may be configured to output an alert to the user of the system proximate to the plaque burden graphic 824 on the display (e.g., the display 132) when the distal and/or proximal end of a planned stent (e.g., as shown by the indicators 816 and 820 of FIG. 8) are within healthy regions of the vessel. The processor circuit 510 may be configured to output a second, separate alert if the distal and/or proximal ends of a planned stent are not within healthy regions of the vessel. In this way, the system 100 advantageously provides the user with feedback regarding the planned placement of a stent. As a result, the likelihood of accurately placing a treatment device, such as a stent, is greatly increased.

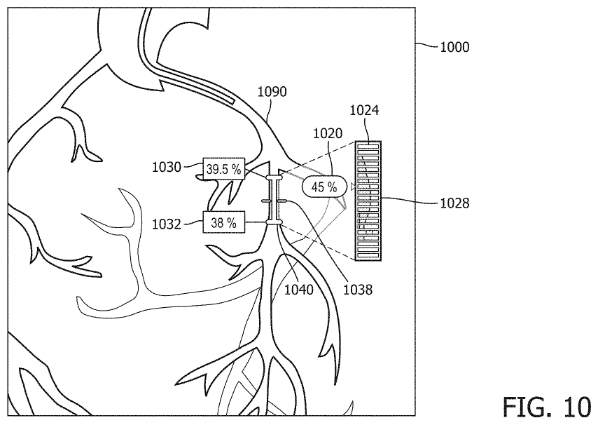

Using plaque burden as an input to determine optimal stent edge positions improves long term outcomes for patients. Percutaneous coronary intervention (PCI) stent planning can be used to create optimal stent landing zones for lesions based off of plaque burden levels at a stent's proximal and/or distal edges. Aspects of the invention may also be used in a peripheral context. By showing the plaque burden value at a proximal and distal cursor combined with a length indication between the two, an interventionist can insure stent size and location are optimized to lower long term stent complications. The intent of stent placement may be to not land the stent in regions of high plaque or region with a high plaque burden. Furthermore, automatic display of plaque burden indicators can be correlated with an angiogram image to increase their utility during stent planning, as will be described in greater detail with reference to FIGS. 10-12.

FIG. 10 is a diagrammatic view of an extraluminal image 1000 including a plaque burden indication 1028, according to aspects of the present disclosure. FIG. 10 may include a depiction of the extraluminal image 1000 with a depiction of a recommended stent in conjunction with the stylized plaque burden ILD 1028. As shown in FIG. 10, the extraluminal image 1000 may be overlaid with or may include any suitable measurements or data, including any of the information, measurements, data, or graphical representations shown and described with reference to FIG. 8 and/or FIG. 9.

In some embodiments, because the received IVUS imaging data from a particular imaging procedure is coregistered to a roadmap extraluminal image (as described with reference to FIG. 7), any of the IVUS related data, or IVUS derived data, including but not limited to a vessel wall identification, vessel diameter, vessel area, lumen boundary identification, lumen diameter, lumen area, plaque burden value, or any other data may be associated with positions along an extraluminal image, like the image 1000 shown in FIG. 10.

The image 1000 may be any suitable extraluminal image. For example, the image 1000 may be an x-ray image such as an angiogram image or a fluoroscopy image. In some embodiments, the extraluminal image 1000 may be an x-ray image obtained with contrast. In some embodiments, the image 1000 may be obtained without contrast. In some embodiments, the image 1000 may be a stylized version of an extraluminal image. For example, the image 1000 may be measurement based or may be a reconstruction of an extraluminal image.

The image 1000 may include a depiction of a stent 1040. The location of the stent 1040 may correspond to the location of the recommended stent shown in FIG. 8. In addition, the image 1000 may include a longitudinal plaque burden indication or graphic 1024. The longitudinal plaque burden graphic 1024 may include any of the features of the longitudinal plaque burden graphic 824 described with reference to FIG. 8 and FIG. 9. For example, the longitudinal plaque burden graphic 1024 may include multiple segments representative of one or more IVUS images. The multiple segments may be visually differentiated from one another to convey measurements or data relating to the IVUS images acquired at the locations shown in the extraluminal image 1000.

As shown, the longitudinal plaque burden graphic 1024 shown may correspond to the length of the vessel identified by the stent 1040 within the image 1000. The longitudinal plaque burden graphic 1024 may include an indicator 1020. The indicator 1020 may identify which segment of the graphic 1024 corresponds to the selected location along the vessel 1090 shown by the indicator 1038. For example, a location may be shown by the indicator 1038 along the vessel in the image 1000. This indicator 1038 may identify any suitable location. In some embodiments, this location may correspond to a region of interest including a location of minimum lumen diameter or minimum lumen area or other region of interest. In some embodiments, this location may be automatically identified and the indicator 1038 may be automatically generated and placed at this location by the processor circuit 510. In other embodiments, the user may select the location of the indicator 1038 with a user input. In some embodiments, the location of the indicator 1038 may not correspond to a region of interest but may be the location selected by a user of the system 100.

The indicator 1020 may display the plaque burden associated with the location 1038 and the corresponding segment 1028 of the longitudinal plaque burden graphic 1024.

In some embodiments, the depiction of the stent 1040 may also include indicators 1030 and 1032. The indicators 1030 and 1032 may display to the user the plaque burden values of the distal and proximal ends of the stent 1040 shown.

As described with reference to FIG. 8, in some embodiments, the circuit 510 may be configured to receive a user input moving the locations of either the stent or the ends of the stent 1040 within the image 1000. For example, the user may select a portion of the stent 1040 causing the stent to move in a proximal or distal direction along the vessel. As the stent 1040 is moved to a different location along the vessel, the indicators 1030 and/or 1032 may update to display the plaque burden values associated with the new positions of the distal and proximal ends of the stent 1040. Similarly, the longitudinal plaque burden graphic 1024 may be moved in conjunction with the stent 1040. The segments of the graphic 1024 may also be updated to match the positions of the stent 1040.

In some embodiments, the circuit 510 may be configured to receive a user input moving the locations of one of the ends of the stent 1040 within the image 1000. As one end of the stent 1040 is moved to a different location along the vessel, the respective indicator 1030 or 1032 may update to display the plaque burden value associated with the new position. Similarly, the associated end of the longitudinal plaque burden graphic 1024 may be moved in conjunction with the stent 1040 and the longitudinal plaque burden graphic 1024 may be extended or shortened accordingly. The segments (e.g., segments similar to the segments 910 of the longitudinal plaque burden graphic 824 described with reference to FIG. 9) of the graphic 1024 may also be updated to match the positions of the stent 1040.

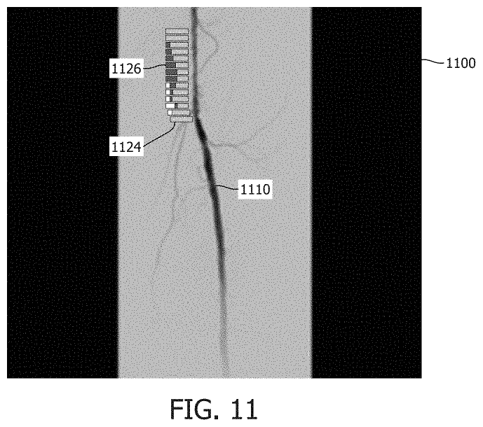

FIG. 11 is a diagrammatic view of an extraluminal image 1100 including a plaque burden indication 1124, according to aspects of the present disclosure. In some embodiments, the extraluminal image 1100 may be a venogram image. For example, the image 1100 may be an x-ray image obtained with contrast introduced to the patient vasculature. Like the image 1000 of FIG. 10, the extraluminal image 1100 may be overlaid with or may include any suitable measurements or data, including any of the information, measurements, data, or graphical representations shown and described with reference to FIG. 8 and/or FIG. 9.

As shown in FIG. 11, the extraluminal image 1100 over which various coregistered data is overlaid may be any suitable extraluminal image. For example, the extraluminal image 1100 may be a venogram. The extraluminal image 1100 may be an image obtained while a contrast agent was introduced to the vessel such that the vessel is visible within the image 1100. In some embodiments, the extraluminal image 1100 may be one image received from a series of extraluminal images received sequentially or in chronological order.

The longitudinal plaque burden graphic 1124 may be similar to the longitudinal plaque burden graphic 1024 of FIG. 10. In some embodiments, the segments 1126 of the ILD 1124 may be positioned adjacent to the vessel 1100 as shown.

In some embodiments, the extraluminal image 1100 shown in FIG. 11 may be a roadmap image. For example, the image 1100 may be similar to the image 711 of FIG. 7 in that it may be used by the processor circuit 510 to determine a pathway of a device through the vessel for coregistration purposes.

As shown in FIG. 11, the longitudinal plaque burden graphic 1124 may include multiple segments 1126. The segments 1126 may be substantially similar to the segments 910 of the longitudinal plaque burden graphic 824 described with reference to FIG. 9. For example, the segments 1126 may each correspond to one or more IVUS images obtained during an IVUS imaging and/or coregistration procedure. As shown in FIG. 11, each segment 1126 may include various regions visually differentiated from one another. As described with reference to FIG. 9, the relative sizes of these regions may indicate percentages of a vessel area associated with various materials structures of a vessel lumen.

It is additionally noted that the segments 1126 of the longitudinal plaque burden graphic 1124, as well as any of the segments described herein including segments 910 of the graphic 824 and the segments 1028 of the graphic 1024, may be arranged in any suitable manner. For example, as shown in FIG. 9, the segments 910 may be aligned with one another vertically or horizontally. The segments 910 may be outlined by a border. However, as shown in FIG. 11, in some embodiments, the segments 1126 may not be aligned with each other, but rather aligned with the vessel 1110.

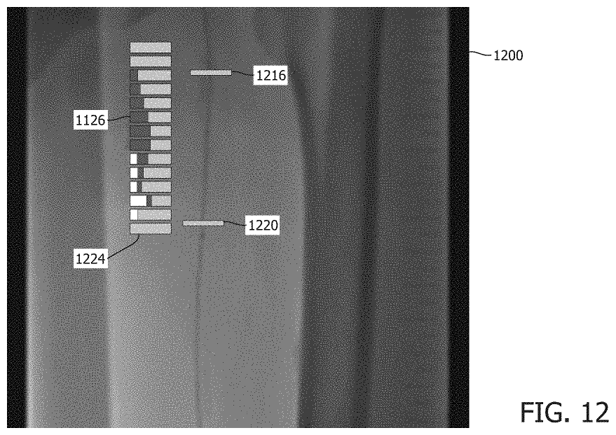

FIG. 12 is a diagrammatic view of an extraluminal image 1200 including a plaque burden indication 1224, according to aspects of the present disclosure. FIG. 12 may correspond to a graphic displayed to a user of the system 100 during a stent deployment phase of a treatment procedure.

In some embodiments, various data or measurements obtained during an imaging phase of a treatment procedure (i.e., plaque burden values, individual IVUS images, vessel wall and/or lumen boundary identification, etc.) may be displayed to a user overlaid on an extraluminal image, such as the image 1200, during deployment of a stent. For example, as shown in FIG. 12, a longitudinal plaque burden indication 1224 may be displayed adjacent to a vessel in which a stent is being deployed. The longitudinal plaque burden indication 1224 may be similar to the plaque burden graphic 1124, the graphic 824, or the graphic 1024 previously described. The display of the longitudinal plaque burden graphic 1224 during deployment of a stent may assist a user in placing the stent in an optimal position. Various aspects of displaying data and measurements from an imaging phase of a treatment procedure during a treatment procedure for guidance may include one or more features described in U.S. Provisional Application No. 63/187,961, filed May 13, 2021, and titled “INTRALUMINAL TREATMENT GUIDANCE FROM PRIOR EXTRALUMINAL IMAGING, INTRALUMINAL DATA, AND COREGISTRATION”, and/or U.S. Provisional Application No. 63/090,638, filed Oct. 12, 2020, and titled “EXTRALUMINAL IMAGING BASED INTRALUMINAL THERAPY GUIDANCE SYSTEMS, DEVICES, AND METHODS”, each of which are herein incorporated by reference in its entirety.

In some embodiments, the image 1200, and any other extraluminal images obtained during the treatment procedure, may need to be obtained at the same angle and magnification of the extraluminal imaging device as was used by the extraluminal imaging device during the initial imaging or coregistration procedure. In some embodiments, intravascular data which was coregistered to locations along a guidewire or the vessel imaged may be associated with various landmarks or features of the anatomy that may be recognized with image processing in subsequent treatment procedures. The positions of these identified anatomy features can be used as part of coregistering the intravascular imaging data from an imaging phase to locations along a guidewire or the vessel in one or more extraluminal images during a live stent deployment procedure.

An optimal stent placement position may correspond to one in which both ends of the stent are positioned in regions of little to no plaque burden. For example, the processor circuit 510 may determine or receive a threshold plaque burden value. The processor circuit 510 may ensure that both ends of a placed stent are positioned at regions at which the plaque burden level is less than the threshold value. In some embodiments, during a stent deployment procedure, the processor circuit 510 may be configured to generate one or more alerts, including a visual alert, audio alert, or haptic alert, when one of the ends of stent are positioned at regions with plaque burdens higher than the threshold value.

As shown in FIG. 12, the processor circuit may also generate various visual indicators, such as indicators 1216 and 1220 corresponding to the proximal and distal ends of a stent. These indicators may be of any suitable appearance.

During a stent deployment procedure, the position and size of the longitudinal plaque burden graphic 1224 may be continually updated to match the regions of the vessel observed in the image 1200. In some embodiments, the image 1200 may be substantially similar to an extraluminal image obtained during a prior imaging procedure. In some embodiments, the image 1200 may be similar to, for example, a roadmap image (e.g., the image 711 of FIG. 7) obtained during a pullback procedure and to which various IVUS images and corresponding data are coregi stered.

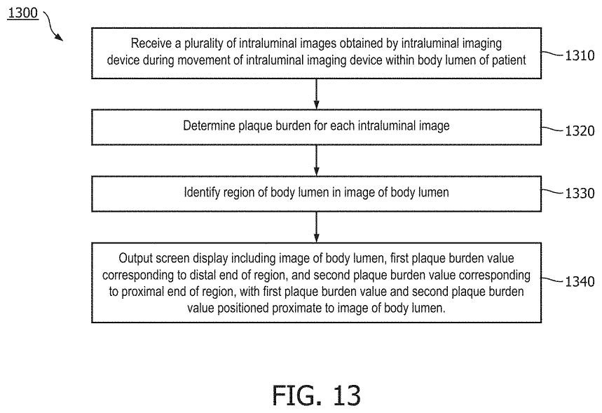

FIG. 13 is a flow diagram of a method 1300 of generating a plaque burden indication and a longitudinal view of a vessel, according to aspects of the present disclosure. The method 1300 may describe an automatic segmentation of a vessel to detect segments of interest using co-registration of invasive physiology and x-ray images. As illustrated, the method 1300 includes a number of enumerated steps, but embodiments of the method 1300 may include additional steps before, after, or in between the enumerated steps. In some embodiments, one or more of the enumerated steps may be omitted, performed in a different order, or performed concurrently. The steps of the method 1300 can be carried out by any suitable component within the diagnostic system 100 and all steps need not be carried out by the same component. In some embodiments, one or more steps of the method 1300 can be performed by, or at the direction of, a processor circuit of the diagnostic system 100, including, e.g., the processor 560 (FIG. 5) or any other component.

Aspects of the invention may receive as inputs: IVUS pullback data of a full vessel; algorithms to accurately determine intraluminal border and EEL (e.g., a vessel wall); and visual representations of plaque burden at the proximal and/or distal markers. In that regard, this invention relates to automated visual output of plaque burden and tissue type that is co-registered to an ILD and an angiographic roadmap.

FIG. 13 is a flow diagram of a method 1300 of automatically segmenting a vessel and generating a treatment plan for the vessel based on coregistration of physiology data and extraluminal data, according to aspects of the present disclosure. The method 1300 may describe an automatic segmentation of a vessel to detect segments of interest using co-registration of invasive physiology and x-ray images. As illustrated, the method 1300 includes a number of enumerated steps, but embodiments of the method 1300 may include additional steps before, after, or in between the enumerated steps. In some embodiments, one or more of the enumerated steps may be omitted, performed in a different order, or performed concurrently. The steps of the method 1300 can be carried out by any suitable component within the diagnostic system 100 and all steps need not be carried out by the same component. In some embodiments, one or more steps of the method 1300 can be performed by, or at the direction of, a processor circuit of the diagnostic system 100, including, e.g., the processor 560 (FIG. 5) or any other component.

At step 1310, the method 1300 includes receiving multiple intraluminal images obtained by the intraluminal imaging device during the movement of the intraluminal imaging device within a body lumen of a patient. In some aspects, step 1310 may include receiving multiple IVUS images obtained by the IVUS imaging device during the movement of the IVUS imaging device within a blood vessel of a patient.

At step 1320, the method 1300 includes determining a plaque burden for each of the multiple intraluminal images. In some aspects, step 1320 may include determining a plaque burden for each of the multiple IVUS images.

At step 1330, the method 1300 includes identifying a region of the body lumen in an image of the body lumen. The image of the body lumen may be any suitable image, including a longitudinal view of the body lumen. A longitudinal view of the body lumen may be an ILD. The longitudinal view may be generated by a processor circuit (e.g., the processor circuit 510 of FIG. 5) based on the multiple intraluminal images. In some aspects, step 1330 may include identifying a region of the blood vessel in an image of the blood vessel. The processor circuit may also be configured to generate a plaque burden indicator corresponding to the region of the blood vessel.

At step 1340, the method 1300 includes outputting, to a display in communication with the processor circuit, a screen display. The screen display may include the image of the body lumen, a first plaque burden value corresponding to a distal end of the region, and a second plaque burden value corresponding to a proximal end of the region. The first plaque burden value and the second plaque burden value are positioned proximate to the image of the body lumen. The first plaque burden value and the second plaque burden value may be positioned proximate to the image of the body lumen in that the first plaque burden value and/or the second plaque burden value may be overlaid over the image, beside the image, for example, to the right or left of the image or above or below the image, or positioned in any other way relative to the image of the body lumen. In some aspects, step 1340 may include outputting, to a display in communication with the processor circuit, a screen display including the image of the blood vessel, a first plaque burden value corresponding to a distal end of the region, a second plaque burden value corresponding to a proximal end of the region, and the plaque burden indicator.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an intraluminal imaging device, wherein the processor circuit is configured to:
receive a plurality of intraluminal images obtained by the intraluminal imaging device during the movement of the intraluminal imaging device within a body lumen of a patient;
determine a plaque burden for each of the plurality of intraluminal images;
identify a region of the body lumen in an image of the body lumen;
output, to a display in communication with the processor circuit, a screen display comprising:
the image of the body lumen;
a first plaque burden value corresponding to a distal end of the region; and
a second plaque burden value corresponding to a proximal end of the region, wherein the first plaque burden value and the second plaque burden value are positioned proximate to the image of the body lumen.

2. The system of claim 1, wherein the image of the body lumen comprises a longitudinal view of the body lumen.

3. The system of claim 2, wherein the processor circuit is further configured to generate a plaque burden indicator corresponding to the region.

4. The system of claim 3, wherein the plaque burden indicator includes one or more segments, each segment corresponding to one or more intraluminal images of the plurality of intraluminal images.

5. The system of claim 4, wherein each segment includes one or more regions, the visual appearance of the one or more regions corresponding to a cross-sectional area of the body lumen or a cross-sectional area of plaque.

6. The system of claim 3, wherein the plaque burden indicator is positioned within the screen display such that the plaque burden indicator is aligned with the region in the longitudinal view.

7. The system of claim 1, wherein the processor circuit is further configured to determine a recommended position for a treatment device based on the plaque burden of one or more of the plurality of intraluminal images.

8. The system of claim 7, wherein the first plaque burden value corresponds to a distal end of the treatment device and the second plaque burden value correspond corresponds to a proximal end of the treatment device.

9. The system of claim 2, wherein the processor circuit is further configured to:
receive, by an input device, a user input moving at least one of the proximal end of the region or the distal end of the region to a different location; and
in response to the user input, update at least one of the first plaque burden value or the second plaque burden value based on the different location.

10. The system of claim 1,
wherein the processor circuit is further configured for communication with an extraluminal imaging device,
wherein the image of the body lumen comprises the extraluminal image.

11. The system of claim 10, wherein the processor circuit is further configured to coregister the plurality of intraluminal images and the plaque burden for each of the plurality of intraluminal images to corresponding positions within an extraluminal image.

12. The system of claim 11, wherein the processor circuit is further configured to generate a plaque burden indicator corresponding to the region.

13. The system of claim 12, wherein the plaque burden indicator includes one or more segments, each segment corresponding to one or more intraluminal images of the plurality of intraluminal images.

14. The system of claim 13, wherein each segment includes one or more regions, the visual appearance of the one or more regions corresponding to a cross-sectional area of the body lumen or a cross-sectional area of plaque.

15. The system of claim 14, wherein the plaque burden indicator is positioned within the screen display such that the plaque burden indicator aligns with the region in the extraluminal image.

16. A method, comprising:
receiving, with a processor circuit in communication with an intraluminal imaging device, a plurality of intraluminal images obtained by the intraluminal imaging device during a movement of the intraluminal imaging device within a body lumen of a patient;
determining, with the processor circuit, a plaque burden for each of the plurality of intraluminal images;
identifying, with the processor circuit, a region of the body lumen in an image of the body lumen;
outputting, to a display in communication with the processor circuit, a screen display comprising:
the image of the body lumen;
a first plaque burden value corresponding to a distal end of the region; and
a second plaque burden value corresponding to a proximal end of the region, wherein the first plaque burden value and the second plaque burden value are positioned proximate to the image of the body lumen.

17. A system, comprising:
an intravascular imaging device; and
a processor circuit configured for communication with the intravascular imaging device, wherein the processor circuit is configured to:
receive a plurality of intravascular images obtained by the intravascular imaging device during the movement of the intravascular imaging device within a blood vessel of a patient;
determine a plaque burden for each of the plurality of intravascular images;
identify a region of the blood vessel in an image of the blood vessel;
generate a plaque burden indicator corresponding to the region of the blood vessel;
output, to a display in communication with the processor circuit, a screen display comprising:
the image of the blood vessel;
a first plaque burden value corresponding to a distal end of the region;
a second plaque burden value corresponding to a proximal end of the region; and
the plaque burden indicator.
